(19) United States
(12) Patent Application Publication
(10) Pub. No.: US 2019/0000834 A1
(43) Pub. Date: Jan. 3, 2019

(54) INJECTABLE COMPOSITION AND A METHOD OF TREATMENT OF THE PHYSICAL ADDICTION

(71) Applicant: VERITA RESEARCH PTE LTD, Caringbah, NSW (AU)

(72) Inventors: Grace CONSONI, North Sydney, NSW (AU); Daryll Ian KNOWLES, North Sydney, NSW (AU)

(21) Appl. No.: 16/062,840

(22) PCT Filed: Dec. 16, 2016
(86) PCT No.: PCT/AU2016/051255

§ 371 Date: Jun. 15, 2018

(30) Foreign Application Priority Data
Dec. 17, 2015 (AU) 2015905238

(51) Int. Cl. A61K 31/485; A61K 9/00; A61K 9/06; A61K 47/34

(52) U.S. Cl. CPC A61K 31/485; A61K 47/34; A61K 9/06; A61K 9/0019

(57) ABSTRACT

An injectable liquid composition for the treatment of a physical addiction includes naltrexone in the amount of 5%-95% of the total weight of the composition; at least two co-polymers in the amount of between 5%-95% of the total weight of the composition; and a biocompatible water miscible organic solvent in the amount of 5% to 50% of the total weight of the composition. The composition is adapted to form a gel-like mass when contacted with an aqueous environment and to dissolve at a predetermined rate to release a therapeutic naltrexone dose over a predetermined period of time.

### FIELD OF THE INVENTION

The present invention relates to treatment of a physical addiction, and, in particular, to an injectable composition and method of delivering same in the treatment of the physical addiction of a person.

### FIELD OF THE INVENTION

The invention has been developed primarily with respect to an injectable composition delivery means for the controlled release of Naltrexone in the treatment of alcohol methamphetamine and opioid addiction and will be described hereinafter with reference to these particular applications. However, it will be appreciated that the invention is not limited to this particular field of use and is applicable in the treatment of the physical addiction of non-opioid pharmaceuticals for compulsive behaviours such as gambling.

Physical addictions are well known in societies across the world. Physical addiction or dependence as it may be sometime termed results from the use of a drug or other stimuli that provides rewards for consuming the drug or being exposed to the stimuli. It is usual from those suffering a physical addiction to suffer or undergo withdrawal symptoms when they cease using the drug or being exposed to the stimuli. In the case of alcohol, for example, this is a wide spread problem worldwide.

In the Australian community, alcoholism or a physical dependence for an addiction on alcohol is a problem across all socioeconomic groups and affects all aspects of the Australian community in one form or another. The treatment for persons suffering from alcoholism or on a dependence on alcohol varies depending on the practitioner. Currently, the known opioid antagonist Naltrexone is used in the treatment of alcoholism. Currently, the Naltrexone is delivered orally in tablet form and this requires daily or multiple daily doses. In the alternative, solid surgically implanted pellets are used in the treatment of alcoholism. The Naltrexone implant includes a coating in which the Naltrexone is allowed to be gradually released over a period, for example, three months which overcomes the problem of requiring daily tablet dosing.

Regular tablet dosing requires close supervision of patients and there needs to be a genuine desire for the patient for themselves want to change their alcohol seeking behaviour on an ongoing basis. This renders the regular complicated tablet dosing protocols relatively unsuccessful in practice. The use of Naltrexone can be positive in the treatment of alcohol addiction or dependence insofar as it can reduce a user's craving to consume alcohol and when they do, the Naltrexone limits physiological reward affects provided by consumption.

In Australia, the regular tablet dosing regime has found in practice to be generally unsuitable for those living in remote areas or for those without regular local medical care or supervision ensuring the tablets are taken and support provided to abstain from alcohol consumption. This is because the patient is required to make a daily decision as to whether to continue the treatment in taking the Naltrexone tablets or return to the drug seeking behaviour with its innate rewards. It appears to be the case that faced with this decision, for patients in remote areas of Australia compliance in conforming to the tablet dosing regime is relatively very poor.

To overcome the disadvantages in the tablet dosing regime, the solid surgically implanted Naltrexone pellets require surgical conditions and a qualified doctor to perform the surgery of incising the patient, implanting the pellets/s and closing the wound. Whilst this overcomes the need for daily tablet dosing, it is well known there is a lack of availability of suitably qualified doctors in regional Australia let alone remote areas. It is known that frequent secondary infections of wounds from the implanted pellets occur and there is a tendency for patients in remote areas to pick at their wounds and in some cases remove the solid surgically implanted pellets. This may be for cultural reasons or general discomfort with having a solid mass implanted.

Naltrexone is an opioid receptor antagonist with a short elimination half-life of about 4 hour (it's active metabolite 6-beta naltrexol has a half-life of approximately 13 hours). The primary indications for the use of Naltrexone are the management of opioid and alcohol addiction. Naltrexone is currently available as Australian Therapeutic Goods registered oral tablets and a non-registered solid pellet. The administration of a series of intramuscular injections is preferred to single and multiple surgically implanted solid pellet or a complicated daily requirement of oral dosing.

Daily oral tablets have a complex dosing regimen with maintenance dose if no withdrawal signs occur, 50 mg orally once a day or an alternative dose schedule: (to improve compliance) 50 mg orally on week days and 100 mg orally on Saturday; or 100 mg orally every other day; or 150 mg orally every third day, for example. The doses in oral tablet must be relatively high to account for degradation from the hepatic first pass effect.

Opioids are psychoactive drugs that produce their pharmacological actions, including analgesia, by acting on receptors located on neuronal cell membranes. The presynaptic action of opioids to inhibit neurotransmitter release is considered to be their major effect in the nervous system. Recent advances in the molecular biology of opioid receptors has confirmed that there are 3 types of opioid receptor, m, d and k. All are coupled to intracellular mechanisms via G-proteins (see Opioids—mechanisms of action Loris A. Chahl, Associate Professor, Discipline of Clinical Pharmacology, Faculty of Medicine and Health Sciences, University of Newcastle, Newcastle, N.S.W.). Opioids have sedative, hypnotic, smooth muscle relaxing actions and are used for anaesthesia and the treatment of pain.

Opioid antagonists reverse the effects of opioids by competitively inhibiting opioid binding to the Opioid receptor. Opioid antagonists are used for a complete or partial reversal of the effects of opioids and for the management of opioid addiction.

Naltrexone ((4R,4aS,7aR,12bS)-3-(cyclopropylmethyl)-4a,9-dihydroxy-2,4,5,6,7a,13-hexahydro-1H-4,12-methanobenzofuro [3,2-e] isoquinoline-7-one) is opioid antagonist that has been found to be effective in the complete or partial reversal of the effects of opioids and for the treatment of opioid overdose. Naltrexone has also been shown to be effective in the treatment of stimulant substance abuse (WO 2006/115743), neuropathic pain (WO 2006/115302), and alcohol dependence (WO 2002/056964).

Naltrexone has also been shown to be beneficial in the treatment of methamphetamine dependence. There is evidence that the endogenous opioid system plays a role in the reinstatement of methamphetamine seeking behavior and behavioral sensitization (see Chiu C T, Ma T, Ho I K. Attenuation of methamphetamine-induced behavioural sensitization in mice by systemic administration of naltrexone. Brain Res Bull. 2005; 67:100-9. [PMC free article] [PubMed]) in methamphetamine self-administering animals. Naltrexone, an opioid antagonist, attenuated cue—but not drug-induced methamphetamine in animals (Anggadiredja K, Sakimura K, Hiranita T, Yamamoto T. Naltrexone attenuates cue—but not drug-induced methamphetamine drug-seeking: a possible mechanism for dissociation of primary and secondary reward. Brain Res. 2004; 1021:272-6. [PubMed]).

In humans, naltrexone may reduce the reinforcing effects of amphetamine via modulation of the opioid system (Jayaram-Lindstrom N, Wennberg P, Hurd Y L, Franck J. Effects of naltrexone on the subjective response to amphetamine in healthy volunteers. J Clin Psychopharmacol. 2004; 24:665-9. [PubMed]). Naltrexone 50 mg along with CBT was evaluated in a 12 week open clinical trial for amphetamine dependence. This medication was well tolerated with moderate rates of compliance (Jayaram-Lindstrom N, Wennberg P, Beck O, Franck J. An open clinical trial of naltrexone for amphetamine dependence: compliance and tolerability. Nord J Psychiatry. 2005; 59:167-71. [PubMed]). In a double-blind placebo-controlled design, naltrexone 50 mg significantly attenuated the subjective effects produced by dexamphetamine in dependent patients. Craving was also significantly blocked (Jayaram-Lindstrom N, Konstenius M, Eksborg S, Beck O, Hammarberg A, Franck J. Naltrexone attenuates the subjective effects of amphetamine in patients with amphetamine dependence. Neuropsychopharmacology. 2008; 33:1856-63. [PubMed]). Moreover, it also was effective in reducing amphetamine use in a recent double-blind, placebo-controlled outpatient clinical trial (Jayaram-Lindstrom N, Hammarberg A, Beck O, Franck J. Naltrexone for the treatment of amphetamine dependence: a randomized placebo-controlled trial. Am J Psychiatry. 2008; 165:1442-8. [PubMed]). Naltrexone therefore appears to be a highly promising medication for amphetamine dependence

Naltrexone has a relatively short terminal half-life of around 4 hours and, therefore, most opioids have longer half-lives than Naltrexone. Accordingly, repeat doses of Naltrexone are often required to prevent the recurrence of opioid addictions symptoms once the initial dose of Naltrexone wears off.

Biodegradable drug delivery systems like fibers, implantable tablets or hard pellets and microspheres have been in use for a long time but suffer from the aforementioned disadvantages.

### GENESIS OF THE INVENTION

The genesis of the invention is a desire to provide an injectable composition and method of using same for the treatment of a physical addiction in a person that will overcome one or more of the disadvantages of the prior art, or to provide a useful alternative.

### SUMMARY OF THE INVENTION

In accordance with a first aspect of the present invention there is provided an injectable liquid composition for the treatment of a physical addiction, the composition comprising:
naltrexone in the amount of 5%-95% of the total weight of the composition;
at least two co-polymers in the amount of between 5%-95% of the total weight of the composition; and
a biocompatible water miscible organic solvent in the amount of 5% to 50% of the total weight of the composition;
wherein the composition is adapted to form a gel-like mass when contacted with an aqueous environment and to dissolve at a predetermined rate to release a therapeutic naltrexone dose over a predetermined period of time.

According to another aspect of the invention there is provided a method of treating a physical addiction in a person, the method comprising the steps of providing a therapeutically useful predetermined volume of the injectable liquid composition according to the first aspect, and injecting same intramuscularly, intradermally or subcutaneously in a person suffering a physical addiction.

It can therefore be seen that there is advantageously provided an injectable Naltrexone composition that once injected forms a soft pellet for use in the treatment of a physical addiction, such as alcohol or other drug dependence delivered by a needle rather than open wound and which is unable to be isolated by feel once implanted.

Advantageously, the use of Naltrexone as an active ingredient in the treatment of a physical addiction allows the injectable composition to be used on multiple or single drug addictions in addition to alcohol and opioid dependence, for example, methamphetamine and cocaine addiction. Yet further advantageously, the injectable composition importantly allows treatment of poly-pharmacy addictions as in practice rarely does a patient present with a single discrete type of addiction but more commonly have a combination of physical substances or stimuli forming the basis of the addiction.

### DETAILED DESCRIPTION

A preferred embodiment of the invention will now be described with reference to an example of the injectable composition (or controlled release depot) for the treatment of alcohol addiction and method of treating a person with a physical addiction according to a preferred embodiment of the invention.

The term “depot” is used in the present specification to encompass any form of localised deposit of a naltrexone wherein the naltrexone is released over a period of time from the localised deposit in-vivo. In this way, the naltrexone composition is adapted or formulated to change its physical state in-vivo from an injectable liquid ex-vivo to a gel-like state upon administration of the formulation. This advantageously forms a gel-like depot of naltrexone composition from which naltrexone is released over a predetermined period of time. In the most preferred embodiment, the gel-like naltrexone composition depot forms at or adjacent the site of administration.

A controlled release depot that releases therapeutic levels of Naltrexone for a period of time makes the hospitalization period shorter and compliance much simpler as the patient isn't faced with the daily decision of whether to take their dose of naltrexone or break out. The treatment continues at home with blood concentrations of Naltrexone been supplied by the depot with no further burden on the patient. Copolymers of polylactid acid (PLA) and polyglycolic acid (PGA) spontaneously form semi-solid depots when a solution of the copolymer in a water miscible organic solvent is injected into water. When a biologically active agent is dissolved or dispersed in the polymer solution an implant containing the drug is formed at the site of injection and the polymer releases the drug in a dual controlled release rate by diffusion and polymer degradation. Compositions containing therapeutic does of Naltrexone according to the preferred embodiment were evaluated as controlled release formulation that supplied Naltrexone at therapeutic levels for up to 30 days. However, it will be appreciated this could be longer or shorter as desired.

Biodegradable drug delivery systems like fibers, implantable tablets or hard pellets and microspheres have been in use for a long time. The major advantage of the injectable single pellet that is soft and dissolvable over time is that a surgery is not required for their implant or removal. Microspheres can be injected and therefore avoid surgical implantation. However, microspheres have also disadvantages like a) inability to remove the dose once injected and b) complicated manufacturing procedures (William J. Lambert, Kendall D. Peck “Development of an in situ forming biodegradable poly-lactide-co-glycolide system for the controlled release of proteins”. Journal of Controlled Release 33 (1995) 189-195) resulting in a relatively expensive end product which may not be affordable to many patients.

Copolymers of polylactid acid (PLA) and polyglycolic acid (PGA) can spontaneously form solid depots when a solution of the copolymer in a water miscible organic solvent is injected into water or a substantially water-based solvent. When the biologically active naltrexone agent is dissolved or dispersed in the polymer solution an implant containing the drug is formed at the site of injection and the polymer releases the drug in a controlled release rate by diffusion and polymer degradation.

The inventors investigated the preferred ratios of PLA/PGA polymers so as to incorporate the active drug pay load in a manner suitable to obtain the desired release rate into the body. Considerations were to create a single semi solid pellet which would break down with dual pharmacokinetic pathways from a single site not leave an open wound and importantly not be able to be felt under the skin by a patient.

A challenge faced by the inventors was to create an injectable formulation which was in liquid state pre injection and upon exposure to body fluids would create a single soft pellet slug. The major advantages here over the current hard pellet technology is the removal of the need for surgical implantation and the risk of secondary infection at the wound site.

Another advantage of the soft injectable pellet is that due to its shorter duration of action (30 days) the managing physician is more in control of the treatment program than when using a 3 or more month hard pellet or a plurality of hard microspheres.

The pliable nature of the soft biodegradable and less invasive nature of the injectable soft pellet of the preferred embodiments also removes the possible need for the addition of immune-suppressants like triamcinolone to stop the body's rejection which the hard pellet requires due to the higher levels of rejection and sterile abscesses observed.

The other advantage of the injectable soft pellet is the cost of manufacturing is significantly lower than hard pellet extrusion and gamma radiation, or the complex and expensive manufacturing process required in the creation of the microspheres.

It will be appreciated that the naltrexone composition of the preferred embodiments is not administered or injected directly into the blood circulation but is delivered intramuscularly, intradermally or subcutaneously. The naltrexone composition depot then releases the naltrexone over a predetermined period of time.

Naltrexone means 17-(cyclopropylmethyl)-4,5α-epoxy-3,14-dihydroxymorphinan-6-one and pharmaceutically acceptable salts, bases and solvates thereof. Pharmaceutically acceptable salts refer to derivatives of naltrexone modified by making acid or base salts thereof. The compounds of this disclosure form acid and base addition salts with a wide variety of organic and inorganic acids and bases and includes the physiologically acceptable salts which are often used in pharmaceutical chemistry. Such salts are also part of this disclosure. Typical inorganic acids used to form such salts include hydrochloric, hydrobromic, hydroiodic, nitric, sulfuric, phosphoric, hypophosphoric and the like. Salts derived from organic acids, such as aliphatic mono and dicarboxylic acids, phenyl substituted alkonic acids, hydroxyalkanoic and hydroxyalkandioic acids, aromatic acids, aliphatic and aromatic sulfonic acids may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, β-hydroxybutyrate, butyne-1,4-dioate, hexyne-1,4-dioate, cabrate, caprylate, chloride, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toleunesulfonate, xylenesulfonate, tartarate, and the like.

Bases commonly used for formation of salts include ammonium hydroxide and alkali and alkaline earth metal hydroxides, carbonates, as well as aliphatic and primary, secondary and tertiary amines, aliphatic diamines. Bases especially useful in the preparation of addition salts include sodium hydroxide, potassium hydroxide, ammonium hydroxide, potassium carbonate, methylamine, diethylamine, and ethylene diamine.

The terms “effective amount” or “therapeutically effective amount” refer to an amount of the naltrexone compound of the preferred embodiments of invention sufficient to provide a benefit in the treatment of an addiction such as to alcohol or opioids. In particular, a therapeutically effective amount means an amount sufficient to provide a therapeutic benefit in-vivo (ie after injection in a subject). Used in connection with an amount of a naltrexone compound of the disclosure, the term preferably encompasses a non-toxic amount that treats the alcohol or opioid addiction.

The term “treating” refers to relieving the disease, disorder, or condition, i.e., causing regression of the disease, disorder, and/or condition. The term “preventing” refers to preventing a disease, disorder, or condition from occurring in a human or an animal that may be predisposed to the disease, disorder and/or condition, but has not yet been diagnosed as having it; and/or inhibiting the disease, disorder, or condition, i.e., arresting its development.

The preferred embodiments of the present invention are concerned with a process for treating a physical addiction in a subject by using a non-intravenous depot injection of a naltrexone composition formulated with a co-polymeric mixture of the aliphatic polyesters polylactic acid (PLA) and polyglycolic acid (PGA).

The chosen PLA/PGA polymer combination of the preferred embodiments result in a naltrexone composition administered in a liquid form that subsequent thereto forms a gel-like depot of the naltrexone composition in-vivo. The term “gel-like”, as used in the context of the preferred embodiments of the present invention, is intended to encompass any relatively high viscosity or density state when compared to the liquid state thereof at injection, e.g., a semi-solid or solid state.

However, polymers used in the preferred embodiment of the invention can include aliphatic polyesters such as polylactides, polyglycolides, polycaprolactones, polydioxanones, polycarbonates, polyhydroxybutyrates, polyalkylene oxalates, polyanhydrides, polyamides, polyesteramides, polyurethanes, polyacetates, polyketals, polyorthocarbonates, polyphosphazenes, polyhydroxyvalerates, polyalkylene succinates, polymalic acids, polyamino acids, polyvinylpyrrolidones, polyethylene glycols, polyhydroxycelluloses, chitins, chitosans and polyorthoesters, or copolymers, terpolymers and combinations and mixtures thereof.

In the preferred embodiments, the copolymers of polylactic acid and polyglycolic acid (poly-lactide and glyclide) spontaneously form gel-like depots when a solution of the copolymer in a water miscible organic solvent is injected into the biological fluids such as sub-cutaneously or intramuiscularly. When naltrexone is dissolved or dispersed in the polymer solution an implant containing the naltrexone forms at or adjacent the site of injection. The polymer releases the naltrexone at a sustained release rate by diffusion and/or polymer degradation.

Most advantageously, once the naltrexone composition of the preferred embodiment is injected, it forms a relatively soft gel. This is not able to be isolated or ‘felt’ as a foreign object under the skin of the patient such as is the case with conventionally hard-pellet naltrexone composition for implantation which can be felt by a person once inserted. Additionally, there is only a needle mark left when administering the naltrexone composition of the preferred embodiment rather than an open wound in the case of hard pellets.

Degradation copolymers of poly-lactide and glyclide can range from one week to over a year, depending on the formulation of the copolymer as well as the method of preparation and formulation. In this way, naltrexone mixed therewith is released over the period the copolymers degrade in-vivo.

Any water-miscible pharmacologically accepted organic solvent can be used such as N-methyl pyrrolidone (NMP), 2-pyrrolidone, dimethyl sulfoxide, glycerol formal, ethanol, propylene glycol or a combination or mixture thereof may be used.

As noted, the release rate of the naltrexone can be altered by the copolymer composition (ratio of poly-lactide to glycolide), copolymer molecular weight, copolymer concentration, loading of naltrexone, solvent, implant size and shape, etc.

It may be desirable to include other ingredients in the composition of the invention; for example a buffering agent, an antioxidant, a free radical scavenger, an antimicrobial agent, and/or a colouring agent. Exact formulations and methods of manufacture will be apparent to those skilled in the art. A number of texts provide assistance in the design and manufacture of pharmaceutical formulations, including Remington's Pharmaceutical Sciences, Mack Publishing Company Co., Easton, Pa.; Remington: The Science and Practice of Pharmacy, Mack Publishing Company Co., Easton, Pa.; Pharmaceutical dosage forms and drug delivery, Ansel et al, 1995, Williams and Wilkins, Malvern, Pa.; and British Pharmacopoeia, The Stationary Office, London.

It will be appreciated that the injected dosage of the naltrexone composition will, of course, vary depending upon known factors, such as the age, health and weight of the recipient; the nature and extent of the symptoms; the kind of concurrent treatment; the frequency of treatment; and the effect desired.

Dosage forms (compositions suitable for administration) can preferably contain from about 10 mg to about 500 mg of naltrexone per unit dose. Preferably, the injected composition releases approximately 5 mg-15 mg per day. In these pharmaceutical compositions, the naltrexone active ingredient (eg naltrexone salt) will ordinarily be present in an amount of about 0.5-95% weight or more preferably about 5-10% weight based on the total weight of the composition.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbents and diluents, surface active agents, suspending agent, tableting binders, lubricants, flavours, and colouring agents.

In a preferred embodiment of the invention, the naltrexone composition includes the components set out in Example 1:

### Example 1 Naltrexone 100 mg/ml of Injectable Liquid Composition; 30 Day Dissolution Period

Naltrexone Base was dissolved in N-methyl pyrrolidone (NMP) in the ratio of 1 gram of natrexone HCl to 11.3 gram of NMP. This was mixed with a racemic mixture of DL lactide/glycolide in the ratio of 47-53 mol % lactide to 53-47 mol % glycolide, and the ratio of the DL mixture is 3.5 gram of DL lactide/glycolide to 1 gram of natrexone base.

### Method of Preparation

Use a glass funnel to add all liquids and solids to a 50 mL amber glass vial.

1) Setup a water bath and heat to 80° C.
2) Weigh Naltrexone, racemic mixture of DL
lactide/glycolide and NMP
3) Add the racemic mixture of DL
lactide/glycolide to the vial
4) Add the Naltrexone to the vial
5) Put a stirrer bar into the vial. Add the NMP and close and seal the vial immediately
6) Place the vial in the water bath and keep stirring until powders have dissolved.
7) Remove vial from the water bath and shake if necessary.
8) When all dissolved, open vial with pliers. Use syringe to draw up the solution. Measure Volume and Q.S. with NMP.
9) Replace volume in the vial and seal the vial. Return the vial to the water bath and stir for 20 mins
8) Using aseptic procedures add via DMSO safe nylon 0.22 micron filter 5.5 mL per vial in the clean room.
9) After each addition, close vials immediately and seal with aluminum cap

### 10) Autoclave

11) Store at room temperature and do not refrigerate.

In the preferred embodiments, the 5 ml of the liquid composition containers 500 mg of naltrexone and the composition is adapted once injected intramuscularly, intradermally or subcutaneously in a person to transform form a liquid to a gel-like mass. The composition preferably is adapted, depending on the intended route of administration, to dissolve at a rate such that a naltrexone level in their blood is maintained between about 2 ng/ml to about 2.5 ng/ml over about a 30 day period. The total volume of the composition will depend on specific characteristics of the patient such as height, weight or genetic background as understood by a medical practitioner.

Initial trials of the composition of the preferred embodiments have been performed to consider the efficacy of the composition when injected into volunteers. Particularly, the volunteers suffered from a variety of different substance addictions including but not limited to opiate, alcohol, nicotine, methamphetamine and polypharmacy involving combinations of all of the above.

The below table is a sample of the demographic and extent of the level of addiction of the patients.

| | | | | | How long has |
|---|---|---|---|---|---|
| | | | | | been |
| Test | Gender | | | | addicted? |
| No. | Male/Female | Age | Race | Family | (years) |
| 1 | Male | 36 | Australia | Laos | 20 |
| 2 | Female | 38 | Australia | Australian | 24 |
| 3 | Male | 55 | Australia | Malta | 34 |
| 4 | Male | 41 | Australia | Italian | 20 |
| 5 | Male | 39 | Australia | Italian | 17 |
| 6 | Male | 70 | Indian | Indian | 30 |
| 7 | Male | 36 | Australia | Italian | 20 |
| 8 | Female | 36 | Greek | Australian | 19 |
| 9 | Male | 35 | Greek | Australian | 19 |
| 10 | Male | 34 | Scottish | Australian | 14 |
| 11 | Female | 33 | Australia | Australian | 16 |
| 12 | Female | 31 | Vietnam | Australian | 11 |
| 13 | Male | 42 | Australia | Australian | 18 |
| 14 | Male | 46 | Australia | Australian | 16 |
| 15 | Male | 32 | Australia | Australian | 15 |
| 16 | Male | 26 | Turkish | Turkey | 6 |
| 17 | Male | 35 | Australia | Greece | 20 |
| 18 | Male | 21 | Australia | Australian | 6 |
| 19 | Male | 46 | Bahrain | Bahrain | 20 |
| 20 | Male | 31 | Australia | Yugoslavia | 10 |
| 21 | Male | 40 | Laos | Laos | 21 |
| 22 | Male | 35 | Vietnam | Vietnam | 17 |
| 23 | Male | 44 | Vietnam | Vietnam | 16 |
| 24 | Male | 36 | Australia | Australia | 12 |
| 25 | Male | 25 | Australia | Australia | 5 |
| 26 | Male | 38 | Australia | Australia | 6 |
| 27 | Female | 37 | Australia | Australia | 3.5 |
| 28 | Male | 35 | Vietnam | tamu | 12 |
| 29 | Male | 32 | Australia | Italian | 17 |
| 30 | Male | 29 | Malaysia | Malaysia | 11 |

Each of the volunteers received the composition of Example 1 of the preferred embodiment intramuscularly by using a 3 mm gluteal injection. Review of the volunteers by a registered medical practitioner subsequent to injection of the composition of Example 1 indicated that at least half of the volunteers did not relapse to recommence use of their substance/s of addiction.

The results of the initial trial have been conclusive that the invention can be effective in the treatment of poly-pharmacy addiction. It is believed by the inventors that more than half of the volunteers above were successfully treated

It has been found that by providing a depot injection of the composition of the preferred embodiment results in the gel-like formation of the composition due to environmental contact with bodily fluids of the subject in need. Once injected, the copolymer of poly-lactide and glyclide significantly reduce the rate of naltrexone release (i.e., delay naltrexone release) removing the need for tablet dosing in any form and is not able to be easily located for felt by a subject have been injected with the naltrexone composition.

As noted, in particularly preferred embodiments, the naltrexone in the composition once injected releases naltrexone at a rate of between 5 mg to 15 mg per day for a 30 day period. This is achieved by use of the composition of the preferred embodiment.

The preferred polymer system results in a naltrexone formulation that is normally present and administered in a liquid form but forms a gel-like depot in vivo. The term “gel-like”, as used in the context of the present invention, is intended to encompass any higher viscosity or density state when compared to a liquid state, e.g., a semi-solid or solid state. The term “depot”, as used in the present invention, is intended to encompass any form of localised deposit of an active agent wherein the active agent is gradually released from the localised deposit. Thus, in the context of the present invention, the naltrexone formulation is able to advantageously change its physical state from a liquid to a gel-like state upon administration of the formulation, thus forming a gel-like depot of naltrexone from which the naltrexone is gradually released over time. Preferably the gel-like depot forms at or near the site of administration.

The foregoing describes only one embodiment of the present invention and modifications, obvious to those skilled in the art, can be made thereto without departing from the scope of the present invention.

The term “comprising” (and its grammatical variations) as used herein is used in the inclusive sense of “including” or “having” and not in the exclusive sense of “consisting only of”.

1. An injectable liquid composition for the treatment of a physical addiction, the composition comprising:
naltrexone in the amount of 5%-95% of the total weight of the composition; at least two co-polymers in the amount of between 5%-95% of the total weight of the composition; and
a biocompatible water miscible organic solvent in the amount of 5% to 50% of the total weight of the composition;
wherein the composition is adapted to form a gel-like mass when contacted with an aqueous environment and to dissolve at a predetermined rate to release a therapeutic naltrexone dose over a predetermined period of time.

2. An injectable liquid composition according to claim 1 including a ratio by weight of about 3 to 4 parts co-polymers to one part naltrexone.

3. An injectable liquid composition according to claim 1 having 100 mg/ml of naltrexone.

4. An injectable liquid composition according to claim 1 wherein the composition is adapted to release about 5 mg to 15 mg naltrexone when in vivo.

5. An injectable liquid composition according to claim 1 including naltrexone in the amount of about 5% to about 10% of the total weight of the composition.

6. An injectable liquid composition according to claim 1 wherein the naltrexone is a pharmaceutically acceptable salt, base or solvate thereof.

7. An injectable liquid composition according to claim 1 wherein the co-polymers are selected from the group consisting of polylactides, polyglycolides, polycaprolactones, polydioxanones, polycarbonates, polyhydroxybutyrates, polyalkylene oxalates, polyanhydrides, polyamides, polyesteramides, polyurethanes, polyacetates, polyketals, polyorthocarbonates, polyphosphazenes, polyhydroxyvalerates, polyalkylene succinates, polymalic acids, polyamino acids, polyvinylpyrrolidones, polyethylene glycols, polyhydroxycelluloses, chitins, chitosans and polyorthoesters, or copolymers, terpolymers and combinations and mixtures thereof.

8. An injectable liquid composition according to claim 1 wherein the co-polymers are polylactic acid and polyglycolic acid.

9. An injectable liquid composition according to claim 1 wherein the composition includes the co-polymers in equal proportions.

10. An injectable liquid composition according to claim 1 wherein the organic solvent is selected from the group consisting of: N-methyl 2-pyrrolidone; 2-pyrrolidone; dimethyl sulfoxide; glycerol; ethanol; propylene glycol; 1, 4 dioxane; and 1, 2 eutanediol; or a combination or mixture thereof.

11. The injectable liquid composition according to claim 1 adapted to be injected intramuscularly, intradermally or subcutaneously in a person.

12. An injectable liquid composition according to claim 1 wherein the composition is adapted to dissolve in-vivo at a predetermined rate to release naltrexone in the amount of 5 mg-15 mg per day.

13. An injectable liquid composition according to claim 1 wherein the composition is adapted to dissolve at a rate so as to maintain a blood concentration of naltrexone of between about 2 ng/ml of blood to about 2.5 ng/ml of blood.

14. An injectable liquid composition according to claim 1 having naltrexone in the amount of about 30% of the total weight of the composition; at least two co-polymers in the amount of about 50% of the total weight of the composition; and organic solvent in the amount of about 10% of the total weight of the composition.

15. A method of treating a physical addiction in a person, the method comprising the steps of providing a therapeutically useful predetermined volume of the injectable liquid composition according to claim 1, and injecting same intramuscularly, intradermally or subcutaneously in a person suffering a physical addiction.

16. A method according to claim 15 wherein the physical addiction includes addiction to one or more of opioids, opiates, alcohol, amphetamines, tropane derivatives including cocaine, and tyramine derivatives including nicotine.

17. The method according to claim 15 or the step of maintaining a blood concentration of naltrexone in a subject of between about 2 ng/ml and 2.5 ng/nl for a period of between 5 days to 90 days.

18. An injectable liquid composition according to claim 2 having 100 mg/ml of naltrexone.

19. An injectable liquid composition according to claim 2 wherein the composition is adapted to release about 5 mg to 15 mg naltrexone when in vivo.

20. An injectable liquid composition according to claim 3 wherein the composition is adapted to release about 5 mg to 15 mg naltrexone when in vivo.
